(19) United States
(12) Patent Application Publication
(10) Pub. No.: US 2005/0143666 A1
(43) Pub. Date: Jun. 30, 2005

(54) Electrocardiograph

(76) Inventor: Akihiko Yanaga, Fujino-cho (JP)

Correspondence Address: WENDEROTH, LIND & PONACK, L.L.P., 2033 K STREET N. W., SUITE 800, WASHINGTON, DC, 20006-1021, US

(21) Appl. No.: 10/510,985

(22) PCT Filed: Mar. 26, 2003
(86) PCT No.: PCT/JP03/03674

(30) Foreign Application Priority Data
Apr. 16, 2002 (JP) 2002-112790

(51) Int. Cl. A61B 5/04

(52) U.S. Cl. 600/509; 600/523

(57) ABSTRACT

The present invention is to provide an electrocardiograph which can measure an electrocardiographic complex easily at occurrence of events in a subject, display it and transmit it. This electrocardiograph is to be constituted of a body case which is held on a chest of a subject and in which an electric circuit is installed inside thereof, a common electrode provided on a back surface which is a chest side of the body case, at least one pair of arm portions extending from the body case, electrocardiographic complex detecting electrodes provided on end portions of the pair of the arm portions respectively, a means for detecting an electrocardiographic complex of the subject based on signals from the electrocardiographic complex detecting electrodes, a display means for displaying the electrocardiographic complex detected by the means for detecting the electrocardiographic complex, a transmission means for transmitting the electrocardiographic complex detected by the means for detecting the electrocardiographic complex, and a switch means for starting to detect, display and transmit the electrocardiographic complex by pressing the common electrode and the electrocardiographic complex detecting electrodes to a chest side so as to make them come in contact with the chest.

### TECHNICAL FIELD

This invention relates to an electrocardiograph which is held on a chest portion of a subject by suspension etc. and can detect, display and transmit an electrocardiogram when a fit occurs in the subject.

### BACKGROUND ART

An electrocardiograph is classified to 12 leads electrocardiograph and a portable Holter electrocardiograph in a large category. The 12 leads electrocardiograph is a large-sized electrocardiograph which is used in medical facilities such as a room for detecting physiology and in which a minute electrocardiogram in a short time at a rest basically is measured.

The Holter electrocardiograph can detect an electrocardiographic complex continuously for 24 hours because it is portable, so that an electrocardiographic complex in activities of daily living except for at a rest can be gained as data. However, there is a problem such that an abnormal electrocardiographic complex can not be gotten easily because almost measured data are normal. Accordingly, many event type electrocardiographs which can measure an electrocardiogram upon occurrence of an oppressive feeling or pains etc. of a chest (events) are commercialized.

However, every prior event type electrocardiograph is a single electrode lead type, so that it is necessary that the subject grips one electrode by his hand and put another electrode to his bared chest for measurement. Thus operation is very nuisance in the case that there is an oppressive feeling in the chest or pains of the chest, and especially, it would be impossible for the subject in whom the events are occurred to measure.

### DISCLOSURE OF THE INVENTION

As described above, the present invention is to provide an electrocardiograph which can measure an electrocardiographic complex easily at occurrence of events in a subject, display it and transmit it.

Accordingly, the present invention is to be constituted of a body case which is held on a chest of a subject and in which an electric circuit is installed inside thereof, a common electrode provided on a back surface which is a chest side of the body case, at least one pair of arm portions extending from the body case, electrocardiographic complex detecting electrodes provided on end portions of the pair of the arm portions respectively, a means for detecting an electrocardiographic complex of the subject based on signals from the electrocardiographic complex detecting electrodes, a display means for displaying the electrocardiographic complex detected by the means for detecting the electrocardiographic complex, a transmission means for transmitting the electrocardiographic complex detected by the means for detecting the electrocardiographic complex, and a switch means for starting to detect, display and transmit the electrocardiographic complex by pressing the common electrode and the electrocardiographic complex detecting electrodes to a chest side so as to make them come in contact with the chest.

Accordingly, due to the present invention, the switch means can be operated by pressing the electrocardiograph by means of a motion similar to a natural motion done by the subject at the occurrence of the events, for instance, a motion for pressing the chest, the electrocardiographic complex can be detected by the electrodes coming in contact with the chest, and the measured result can be displayed and transmitted.

Besides, it is desired that the switch means is constituted of a pair of push-down switches provided in a front surface of the body case, detection, display and transmission of the electrocardiographic complex are started when both of the push-down switches are pushed down at the same time for a specific period. Furthermore, it is also desired that the switch means is constituted of push-down switches provided in the electrodes respectively, and transmission of the electrocardiographic complex are started when all of the push-down switches are pushed down for a specific period by pressing the body case to a human body side.

Moreover, it is desired that the electrodes are non-paste electrodes. It is desired that the non-paste electrode consists of a conductive synthetic resin including carbon-fiber. Thus, sufficient conductivity of the electrodes can be gained without applying the paste.

It is desired that the body case is suspended from a neck by a suspending means. The suspending means is a strap such as strings or chains etc. Besides, the suspending means is detachable to the body case by a detachable means. Thus, it can be prevented that the suspending means twines around the neck of the subject. It is desired that the detachable means is concretely constituted of magnets which are provided in both ends of the strap and plates for attachment consisting of a magnetic material such as iron, stainless steel, etc. and providing on specific positions of side surfaces of the body case respectively.

### BRIEF DESCRIPTION OF THE DRAWINGS

FIG. 1a is a front elevation of an electrocardiograph according to a first embodiment of the present invention and FIG. 1b is a rear elevation of it;

FIG. 2a is a block diagram of an electric circuit housed in a body case and FIG. 2b is a block diagram illustrating one example of a transmission device;

FIG. 3 is a flowchart diagram illustrating operational example of the electric circuit; and

FIG. 4a is a front elevation of an electrocardiograph according to a second embodiment of the present invention and FIG. 4b is a side elevation of it.

### BEST MODE FOR CARRYING OUT THE INVENTION

Hereinafter, a working mode of the present invention is explained by referring drawings.

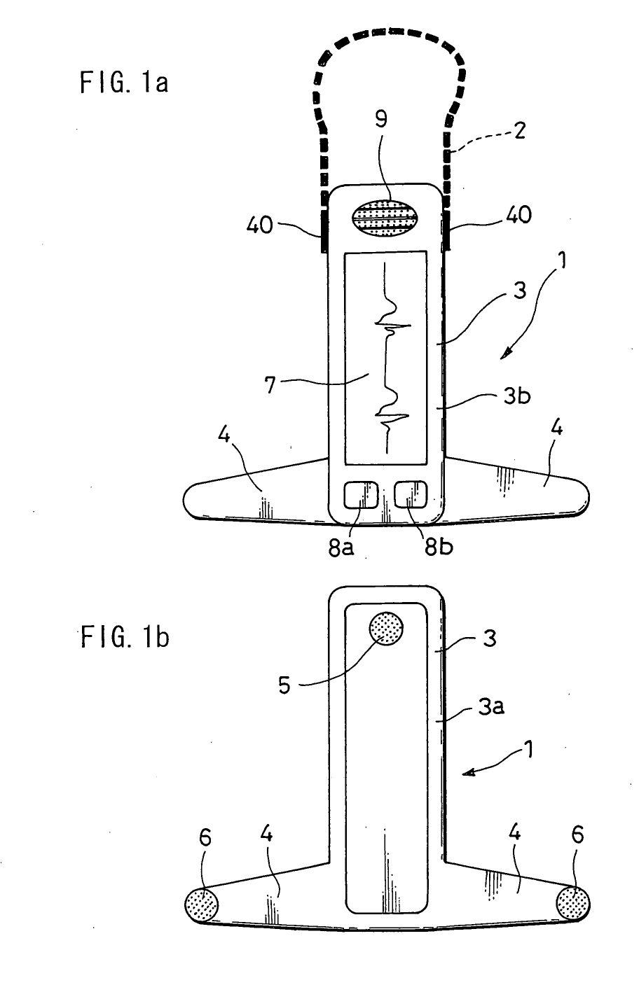

One embodiment of an electrocardiograph according to the working mode of the present invention is shown in FIGS. 1a and 1b. The electrocardiograph 1 is constituted of a body case 3 which is suspended from a neck of a subject by a strap 2 to be held it on a chest of the subject and a pair of electrode arm portions 4 which extend from the body case 3 in both side directions respectively. Besides, a common electrode 5 is provided on a surface 3a in a human body side of the body case 3 and detecting electrodes for electrocardiographic complex 6 are provided on both end portions of the electrode arm portions 4 respectively. Generally speaking, one of the detecting electrodes for electrocardiographic complex 6 is an anode and another of them is a cathode. Furthermore, the common electrode 5 and the detecting electrodes for electrocardiographic complex 6 are non-paste electrodes consisting of conductive synthetic resin material, for instance a synthetic resin material including carbon fiber.

Furthermore, a display screen 7 consisting of a LCD panel, a pair of starting switches 8a and 8b, and a sound indicator 9 including a speaker are provided in a front surface 3b of the body case 3. Besides, in this embodiment, the sound indicator 9 is a buzzer, but it is preferred that notice or alarm etc. is indicated by a voice.

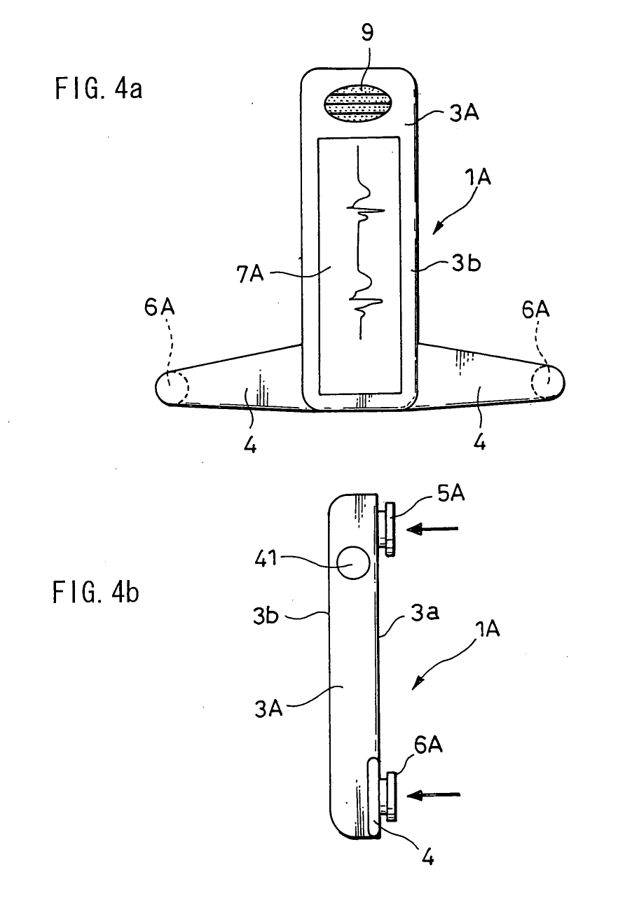

Moreover, the strap 2 comprises cords or chains and magnets for attachment 40 are provided in both ends of it, so that the magnets 40 stick to plates for attachment 41 consisting of magnetic materials such as irons, stainless steels etc. and providing in specific positions in side surfaces of the body case 3 as shown in FIG. 4b to hold the body case 3 in a chest portion of the subject. Furthermore, the magnets 40 can be removed from the plates for attachment when power more than a specific value is applied to the strap 2, so that an accident caused by the strap can be prevented.

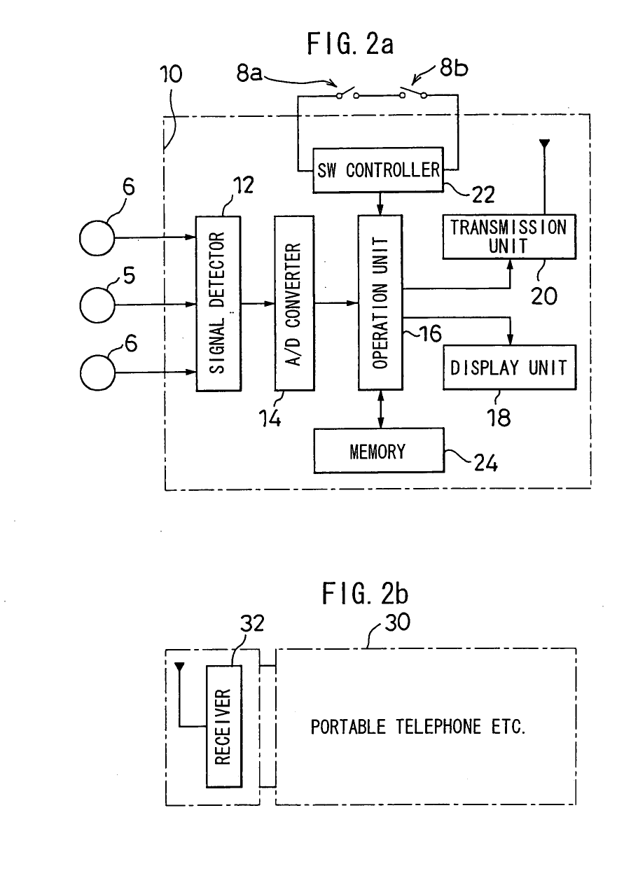

An electric circuit for detecting the electrocardiographic complex from signals detected by the electrodes 5, 6, indicating it and transmitting it is provided inside the body case 3. A block diagram illustrating one embodiment of operation in the electric circuit is shown in FIG. 2a.

In the block diagram, a signal detector 12 detects an analogue signal for the electrocardiographic complex based on change of an electric current value, change of a voltage value, or change of an impedance etc. arisen between the electrodes 5, 6. An A/D converter 14 changes the analogue signal for the electrocardiographic complex to a digital signal which can be operated in an operation unit 16. The operation unit 16 is constituted of at least a central processing unit (CPU), random access memories (RAM), read only memories (ROM) and input/out put ports (I/O) which are not shown in figures, operates input signals along a specific program and changing the input signals to output signals, and further controls the signal detector 12, the A/D converter 14, and a display unit 18 and a transmission unit 20 as follows. Besides it is preferred that a pulse rate is detected from the electrocardiographic complex in the operation unit 16. It is preferred that the electrocardiographic complex operated in the operation unit 16 at a normal condition is stored in a memory unit 24 so as to be able to operate whether the electrocardiographic complex detected at an event time is abnormal and a level of it by comparing with the stored electrocardiographic complex.

Furthermore, a switch controller (SW controller) 22 is connected with the starting switches 8a, 8b and judges whether both of them are pressed in a specific period, for instance 5 seconds, and if so, starting the operation unit 16 and carrying out every operation.

Moreover, the display unit 18 is to display the electrocardiographic complex operated in the operation unit 16 in the display screen 7, but it is preferred that not only the electrocardiographic complex but also the pulse rate and so on can be displayed.

Besides, the transmission unit 20 transmits the electrocardiographic complex by an instruction from the operation unit 16. In this working mode, because there is a case that the subject has a pace maker, the transmission unit 20 is constituted so that the electrocardiographic complex to be transmitted is transmitted to a receiver 32 mounted on a transmission device 30 such as a portable telephone as shown in FIG. 2b with a weak radio wave firstly, and then the transmission device 30 held on a waist or in a bag transmits it to a nurse center or a computer of a doctor for watching. Note that there is a power source such as butteries not shown in figures as the other constitution.

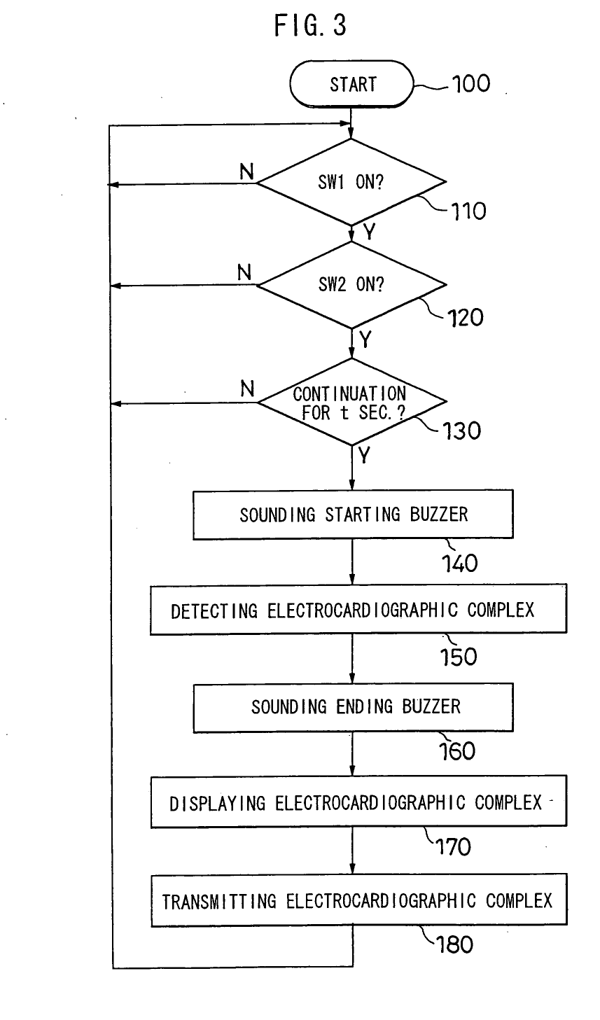

Explaining one embodiment of the operation carried out in the electric circuit constituted as the above along a flowchart diagram starting at a step 100 as shown in FIG. 3, it is judged in a step 110 firstly whether the starting switch 8a (SW1) is pressed or not (ON?) and it is judged in a step 120 whether the starting switch 8b (SW2) is pressed or not (ON?). In these judgments, an operation can be advanced to a step 130 only in a case of it is judged that both starting switches 8a, 8b are pressed at the same time. In the step 130, it is judged whether a condition that the both switches 8a, 8b are pressed is continued for t seconds (for instance, 5 seconds) or not.

In the above steps 110, 120 and 130, only in a case that the both switches 8a, 8b are pressed for t seconds, it is confirmed that the subject wishes to measure the electrocardiographic complex. Namely, it can be judged that an action that the starting switches 8a, 8b on the body case 3 is pressed, for instance, for 5 seconds is done so that the subject presses the electrocardiograph 1 to his chest portion when the event (the pain of the chest etc.) is happened.

Then, the operation is advanced to a next step 140 and a buzzer for starting measurement is sounded in order to inform the starting of the measurement to the subject in the step 140. Then, the operation is advanced to a step 150 and the electrocardiographic complex is detected or the electrocardiographic complex and the pulse rate are detected in the step 150. Then, advancing to a step 160, a buzzer for ending measurement is sounded in the step 160. In this case, for instance, seven short buzzer sounds are sounded to inform the end of the measurement to the subject.

After that, advancing to a step 170, the electrocardiographic complex is displayed via the display unit 18 to the display screen 7 for a specific period. Accordingly, when the subject wants to confirm the electrocardiographic complex, the subject takes out the electrocardiograph 1 from the chest portion and can watch the display screen 7. Besides, there is an advantage that the nurse can confirm the electrocardiographic complex of the subject at the time of occurrence of the event.

Then, advancing to a step 180, the electrocardiographic complex is transmitted to the transmission device 30 via the transmission unit 20. Accordingly, because the nurse or the doctor can confirm the occurrence of the event in the subject and confirm the electrocardiographic complex at the time, suitable treatment can be done.

Hereinafter, explaining an electrocardiograph according to a second embodiment in FIGS. 4a and 4b, some parts in this embodiment which are the same parts in the above mentioned embodiment or have the same effects are marked the same reference numbers to omit the explanations for them.

An electrocardiograph 1A according to the second embodiment is that the starting switches are unitedly installed to electrodes 5A, 6A respectively. Namely, the electrodes 5A, 6A come in contact with a human body by pressing a body case 3A to a side of the human body and are pressed, so that the electrodes 5A, 6A come in contact with the electric circuit to start the operation. Accordingly, structures of the electrodes 5A, 6A become complicated, but the starting switches are turned on automatically by pressing the body case 3A so that the electrodes 5A, 6A come in contact with the human body strongly, as a result, as movement of the subject can be decreased at the occurrence of the events, the measurement becomes easy.

Furthermore, in this embodiment, the starting switches are provided in the electrodes 5A, 6A unitedly, so that there is an effect that the display screen 7A can be made large.

Industrial Applicability

As described above, according to the present invention, because an electrocardiograph is suspended from a neck of a subject by a strap etc. and held on a chest portion of the subject, an electrocardiographic complex is measured, displayed and transmitted by pressing the electrocardiograph held on the chest at occurrence of an event, detection, display and transmission of the electrocardiographic complex become possible in a nature motion of the subject, namely without a special motion of the subject, so that the electrocardiographic complex of the subject at an abnormal condition can be gained exactly.

Besides, because unnecessary measurement can be omitted, a waste of electricity can be decreased, so that a effect that a life of a buttery as a power source can be extended etc. can be gained.

1. An electrocardiograph comprising:
a body case which is held on a chest portion of a subject and in which an electric circuit is provided;
a common electrode that is provided on a back surface which is a chest side of said body case;
a pair of arm portions which extend from said body case;
electrodes for detecting electrocardiographic complex which are provided in both ends portions of said pair of said arm portions respectively;
a detecting means for detecting electrocardiographic complex of said subject based on signals detected by said electrodes for detecting electrocardiographic complex;
a display means for displaying said electrocardiographic complex detected by said detecting means;
a transmitting means for transmitting said electrocardiographic complex detected by said detecting means; and
a switching means for starting detection, display and transmission of said electrocardiographic complex by pressing it to a chest side of said subject so as to make said common electrode and said electrodes for detecting come in contact with said chest of said subject.

2. An electrocardiograph according to claim 1, wherein
said switching means is constituted of a pair of push-down switches which are provided on a front surface of said body case, and makes detection, display and transmission of said electrocardiographic complex start when both of said push-down switches are pushed down at once for a specific period.

3. An electrocardiograph according to claim 1, wherein
said switching means is constituted of push-down switches which are provided in said electrodes respectively, and makes detection, display and transmission of said electrocardiographic complex start when all of said push-down switches are pushed down for a specific period by pressing said body case to a human body side.

4. An electrocardiograph according to claim 1, wherein
said electrodes are non-paste electrodes.

5. An electrocardiograph according to claim 2, wherein
said electrodes are non-paste electrodes.

6. An electrocardiograph according to claim 3, wherein
said electrodes are non-paste electrodes.

7. An electrocardiograph according to claim 1, wherein
said body case is suspended from a neck of said subject by a suspending means.

8. An electrocardiograph according to claim 2, wherein
said body case is suspended from a neck of said subject by a suspending means.

9. An electrocardiograph according to claim 3, wherein
said body case is suspended from a neck of said subject by a suspending means.

10. An electrocardiograph according to claim 4, wherein
said body case is suspended from a neck of said subject by a suspending means.

11. An electrocardiographic according to claim 5, wherein
said suspending means is detachable to said body case by a detachable means.
